(19) United States
(12) Patent Application Publication
(10) Pub. No.: US 2025/0120889 A1
(43) Pub. Date: Apr. 17, 2025

(54) MAGNESIUM HYDROXIDE FACIAL MASK AND METHODS OF MAKING AND USING SAME

(71) Applicant: Dead Sea Bromine Company Ltd., Be'er Sheva (IL)

(72) Inventors: Meyrav ABECASSIS WOLFOVICH, Meitar (IL); Mohammed ABUELHAIGA, Kibbutz Mefalsim (IL); Reuven ROZEN, Beer Sheva (IL); Nikolay FUX, Beer Sheva (IL); Smadar SWISSA, Meitar (IL)

(21) Appl. No.: 18/728,734

(22) PCT Filed: Jan. 16, 2023
(86) PCT No.: PCT/IL2023/050046

§ 371 Date: Jul. 12, 2024

Related U.S. Application Data
(60) Provisional application No. 63/303,533, filed on Jan. 27, 2022.

(51) Int. Cl. A61K 8/19; A61K 8/02; A61K 8/34; A61K 8/37; A61K 8/44; A61K 8/60; A61K 8/73; A61K 8/92; A61K 8/9794; A61Q 19/00

(52) U.S. Cl. CPC A61K 8/19; A61K 8/0212; A61K 8/022; A61K 8/342; A61K 8/345; A61K 8/375; A61K 8/442; A61K 8/602; A61K 8/733; A61K 8/922; A61K 8/9794; A61Q 19/00; A61K 2800/28

(57) ABSTRACT

A facial mask composition comprising: at least 45% magnesium hydroxide; not more than 30% water; and not more than 12% lipids. Related compositions and methods are also disclosed.

### RELATED APPLICATIONS

This PCT application claims the benefit according to 35 U.S.C. § 119(e) of U.S. provisional patent application 63/303,533 filed on Jan. 27, 2022 and having the same title and applicant as the present application, which is fully incorporated herein by reference.

### FIELD OF THE INVENTION

The invention is in the field of cosmetic facial masks.

### BACKGROUND OF THE INVENTION

Facial masks have been known since at least 1875. At that time facial masks were purported to “bleach, purify and preserve the complexion of the skin”.

There are four primary formulations of facial masks:
(I) A rinse mask is a thick cream or paste that is applied and removed by washing with water or soap and water;
(II) A peel mask is a thick cream or paste that solidifies after applicant and can be removed manually (by peeling);
(III) A fabric mask is a piece of material (typically non-woven fabric) impregnated with the mask components which is positioned on the face, patted into place and then removed manually after use; and
(IV) A gel mask is a three-dimensional matrix of a cross linked polymeric material surrounded by an aqueous or oily solution which is typically removed with a washcloth or by washing with water or soap and water.

There are different mask formulations for purposes such as cleaning pores, reduction of oiliness, moisturization of dry skin and increasing skin smoothness. Users often describe the effect of a facial mask as rejuvenating or refreshing.

Application duration varies widely from a few minutes to overnight (sleep masks).

Mask types (II) and (III) are typically removed as a single piece.

### SUMMARY OF THE INVENTION

One aspect of some embodiments of the invention relates to a facial mask containing 50%; 52%; 54%; 56%; 58%; 60%; 62%; 64% or intermediate or greater percentages of magnesium hydroxide. In some embodiments, the mask contains less than 12%; less than 11%; less than 10% or intermediate or lower percentages of lipids. Alternatively, or additionally, in some embodiments the mask does not contain cosmetic clay (e.g. kaolin).

Another aspect of some embodiments of the invention relates to heating water and surfactant (e.g. Cocamidopropyl Betaine (35%)) to form a hot aqueous phase, heating organic components (e.g. Glyceryl Stearate SE and/or Cetearyl Alcohol and/or Cetearyl Glucoside and/or Ceto-stearyl alcohol and/or Glycerin and/or Jojoba oil) to form a hot organic phase, mixing the hot organic phase into the hot aqueous phase and mixing to homogeneity then cooling slightly and adding magnesium hydroxide (e.g. 50% to 64% or more) while mixing. In some embodiments, additives such as Phenoxyethanol and/or Ethylhexylglycerin and/or Aloe vera gel are added at this stage. In some embodiments, the resultant facial mask composition is packaged in wide mouth jars.

Another aspect of some embodiments of the invention relates to applying 50%; 52%; 54%; 56%; 58%; 60%; 62%; 64% or intermediate or greater percentages of magnesium hydroxide to facial skin in order to improve one or more characteristics of the facial skin.

Another aspect of some embodiments of the invention relates to formulation, preparation and use of facial masks that do not contain cosmetic clay.

Another aspect of some embodiments of the invention relates to a dry mix for a peel off facial mask comprising Mg(OH)₂. In some embodiments, the dry mix contains at least 45% Mg(OH)₂.

It will be appreciated that the various aspects described above relate to solution of technical problems associated with producing a magnesium hydroxide facial mask that is stable during storage.

Alternatively or additionally, it will be appreciated that the various aspects described above relate to solution of technical problems related to applying high concentrations of an alkaline material to facial skin without damaging the skin.

In some exemplary embodiments of the invention there is provided a facial mask composition including: (a) at least 45% magnesium hydroxide; (b) not more than 30% water; and (c) not more than 12% lipids. In some embodiments, the composition includes at least 60% magnesium hydroxide. Alternatively or additionally, in some embodiments, the composition includes a surfactant. Alternatively or additionally, in some embodiments, the composition includes at least one member of the group consisting of Glyceryl Stearate SE, Cetearyl Alcohol, Cetearyl Glucoside, Ceto-stearyl alcohol, Glycerin, and Jojoba oil. Alternatively or additionally, in some embodiments, the composition includes at least one member of the group consisting of Phenoxyethanol, Ethylhexylglycerin, and Aloe vera gel. Alternatively or additionally, in some embodiments, the composition is formulated as a member of the group consisting of a rinse mask, a peel mask and a fabric mask. Alternatively or additionally, in some embodiments, the magnesium hydroxide is at least 95% pure. Alternatively or additionally, in some embodiments, the magnesium hydroxide exhibits a maximum loss on drying (MLOD) of ≤2%. Alternatively or additionally, in some embodiments, the magnesium hydroxide exhibits a loss on ignition (LOI) of 27 to 35%. Alternatively or additionally, in some embodiments, the magnesium hydroxide has a bulk density (untapped) of 0.7 g/cc to 1.2 g/cc. Alternatively or additionally, in some embodiments, the magnesium hydroxide has a particle size characterized by a residue on 325 mesh (wet sieve) of ≤12%.

In some exemplary embodiments of the invention there is provided a method including: applying a composition comprising at least 45% magnesium hydroxide to facial skin of a subject. In some embodiments, the composition comprises at least 60% magnesium hydroxide.

In some exemplary embodiments of the invention there is provided a method including: heating water and surfactant to form a hot aqueous phase; heating organic components to form a hot organic phase; and mixing the hot organic phase with the hot aqueous phase and mixing to homogeneity then cooling slightly and adding at least 50% magnesium hydroxide while mixing. In some embodiments, the adding at least 50% magnesium hydroxide includes adding at least 60% magnesium hydroxide. Alternatively or additionally, in some embodiments, the surfactant comprises Cocamidopropyl Betaine. Alternatively or additionally, in some embodiments, the organic components comprise at least one member of the group consisting of Glyceryl Stearate SE, Cetearyl Alcohol, Cetearyl Glucoside, Ceto-stearyl alcohol, Glycerin, Jojoba oil. Alternatively or additionally, in some embodiments, the method includes adding one or more additives selected from the group consisting of Phenoxyethanol, Ethylhexylglycerin, and Aloe vera gel. Alternatively or additionally, in some embodiments, the method includes packaging the resultant facial mask composition in wide mouth jars.

In some exemplary embodiments of the invention there is provided a peel off facial mask composition dry mix including: (a) at least 45% magnesium hydroxide; and (b) other dry ingredients. In some embodiments, the dry mix according includes at least 60% magnesium hydroxide. Alternatively or additionally, in some embodiments the dry mix according includes an alginate. Alternatively or additionally, in some embodiments the dry mix according includes at least one member of the group consisting of calcium sulfate dihydrate, Kelzan, Disodium EDTA and pentasodium triphosphate. Alternatively or additionally, in some embodiments the dry mix according includes microcrystalline cellulose (MCC) and/or silica. Alternatively or additionally, in some embodiments the magnesium hydroxide is at least 95% pure. Alternatively or additionally, in some embodiments the magnesium hydroxide exhibits a maximum loss on drying (MLOD) of ≤2%. Alternatively or additionally, in some embodiments the magnesium hydroxide exhibits a loss on ignition (LOI) of 27 to 35%. Alternatively or additionally, in some embodiments the magnesium hydroxide has a bulk density (untapped) of 0.7 g/cc to 1.2 g/cc. Alternatively or additionally, in some embodiments the magnesium hydroxide has a particle size characterized by a residue on 325 mesh (wet sieve) of ≤12%. In some exemplary embodiments of the invention there is provided a peel off facial mask including a dry mix as set forth hereinabove diluted with water to contain at least 10% magnesium hydroxide. Alternatively or additionally, in some exemplary embodiments of the invention there is provided a peel off facial mask including a dry mix as set forth hereinabove diluted with water to contain at least 1.7% alginate.

In some exemplary embodiments of the invention there is provided a method including: (a) mixing a dry mix containing at least 45% magnesium hydroxide with water at a ratio of dry mix:water between 1:2 and 1:4; and (b) applying the resultant mixture to facial skin as a peel off mask. In some embodiments, the ratio of dry mix:water is between 1:2.5 and 1:3.5. Alternatively or additionally, in some embodiments the method includes peeling the mask off of the facial skin.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is bar graph illustrating a reduction in skin sebum content (μg/cm²) at 28 days as a result of use of a facial mask according to an exemplary embodiment of the invention;

FIG. 2 is bar graph illustrating a reduction in skin pore diameter (μm) at 28 days as a result of use of a facial mask according to an exemplary embodiment of the invention;

FIG. 3 is bar graph illustrating a reduction in skin redness in some subjects at 28 days as a result of use of a facial mask according to an exemplary embodiment of the invention;

FIG. 4 is bar graph illustrating normalizing effect on skin in some subjects at 28 days as a result of use of a facial mask according to an exemplary embodiment of the invention;

FIG. 5 is bar graph illustrating an astringent effect on pores in some subjects at 28 days as a result of use of a facial mask according to an exemplary embodiment of the invention;

FIG. 6 is bar graph illustrating an improvement in skin smoothness in some subjects at 28 days as a result of use of a facial mask according to an exemplary embodiment of the invention; and

FIG. 7 is bar graph illustrating an improvement in protoporfinrin IX distribution in some subjects at 28 days as a result of use of a facial mask according to an exemplary embodiment of the invention.

### DETAILED DESCRIPTION OF EMBODIMENTS

Embodiments of the invention relate to compositions and methods for facial masks which contain magnesium hydroxide (Mg(OH)₂).

Specifically, some embodiments of the invention can be used to improve skin qualities such as softness and/or reduce pore size and/or reduce skin oiliness.

The principles and operation of a composition and/or method according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### Exemplary Ready to Use Composition

In some exemplary embodiments of the invention there is provided a facial mask composition including at least 45% magnesium hydroxide, not more than 30% water and not more than 12% lipids. According to various exemplary embodiments of the invention the composition includes at least 45%; at least 50%; at least 55%; at least 60%; at least 65%; at least 70%; at least 75%; at least 80%; at least 85%; at least 90% or intermediate or greater percentages of magnesium hydroxide. Alternatively or additionally, according to various exemplary embodiments of the invention the composition includes less than 50%; less than 55%; less than 60%; less than 65%; less than 70%; less than 75%; less than 80%; less than 85%; less than 90% or intermediate or lower percentages of magnesium hydroxide. In some embodiments the composition includes at least 60% magnesium hydroxide. In some embodiments, the composition includes 55% to 65% magnesium hydroxide.

In some exemplary embodiments of the invention, the composition includes a surfactant. In some exemplary embodiments of the invention, the surfactant includes cocamidopropyl betaine.

According to various exemplary embodiments of the invention the composition includes group consisting of Glyceryl Stearate SE and/or Cetearyl Alcohol and/or Cetearyl Glucoside and/or Ceto-stearyl alcohol and/or Glycerin and/or Jojoba oil. In some exemplary embodiments of the invention, the composition includes 2 or 3 or more of these ingredients.

Alternatively or additionally, according to various exemplary embodiments of the invention. The composition includes Phenoxyethanol and/or Ethylhexylglycerin and/or Aloe vera gel. In some exemplary embodiments of the invention, the composition includes 2 or all 3 of these ingredients.

According to various exemplary embodiments of the invention the composition is formulated a rinse mask, a peel mask or a fabric mask. In some exemplary embodiments of the invention, peel off formulations are provided as dry powder pre-mixes as explained in greater detail hereinbelow.

A detailed exemplary rinse mask formulation is presented hereinbelow in example 1.

### Exemplary Magnesium Hydroxide Characteristics

In some exemplary embodiments of the invention, the magnesium hydroxide used to prepare the composition, or other compositions described hereinbelow, or used in methods described hereinbelow, has one or more of the following characteristics.

In some exemplary embodiments of the invention, the magnesium hydroxide is at least 95%; at least 95.2%; at least 95.4%; at least 95.6%; at least 95.8%; at least 96%; at least 96.2%; at least 96.4%; at least 96.6%; at least 96.8%; at least 97%; at least 97.2%; at least 97.4%; at least 97.6%; at least 97.8%; at least 98%; at least 98.2%; at least 98.4%; at least 98.6%; at least 98.8%; at least 99%; at least 99.2%; at least 99.4%; at least 96.9%; at least 99.8%; at least 99.9% or substantially 100% pure.

Alternatively or additionally, according to various exemplary embodiments of the invention the magnesium hydroxide exhibits a maximum loss on drying (MLOD) of ≤2.0%; ≤1.8%; ≤1.6%; ≤1.4%; ≤1.2%; ≤1.0%; ≤0.8%; ≤0.6%; ≤0.4%; ≤0.2%; or intermediate or lower percentages.

Alternatively or additionally, according to various exemplary embodiments of the invention the magnesium hydroxide exhibits a loss on ignition (LOI) of 27 to 35% or 29 to 34%.

Alternatively or additionally, according to various exemplary embodiments of the invention the magnesium hydroxide has a bulk density (untapped) of 0.7 g/cc to 1.2 g/cc or 0.8 g/cc to 1.1 g/cc.

Alternatively or additionally, according to various exemplary embodiments of the invention the magnesium hydroxide has a particle size characterized by a residue on 325 mesh (wet sieve) of ≤12%; ≤11.75%; ≤11.5%; ≤11.25%; ≤11.0%; ≤10.75%; ≤10.5%; ≤10.25%; or 10.0% or intermediate or lower percentages.

In some exemplary embodiments of the invention, an increase in purity of the magnesium hydroxide included in the mask contributes to an increase in skin sebum reduction.

Alternatively or additionally, in some exemplary embodiments of the invention, a decrease in MLOD of the magnesium hydroxide included in the mask contributes to an increase in skin sebum reduction.

Alternatively or additionally, in some exemplary embodiments of the invention, a in LOI of the magnesium hydroxide included in the mask contributes to a change in skin sebum reduction.

Alternatively or additionally, in some exemplary embodiments of the invention, a decrease in particle size of the magnesium hydroxide contributes to an increase in magnesium hydroxide surface area. In some embodiments, the increase in magnesium hydroxide surface area contributes to an increase in skin sebum reduction. Alternatively or additionally, in some embodiments a decrease in surface area contributes to an improvement in mask uniformity and/or drying time. In some exemplary embodiments of the invention, use of heavy hydroxide contributes to a decrease in surface area.

### Exemplary Method of Use

In some exemplary embodiments of the invention there is provided a method including applying a composition comprising at least 45% magnesium hydroxide to facial skin of a subject. In some embodiments, the composition is applied as a facial mask and left on for 3, 5, 10, 15, 20, 25 or 30 or intermediate or larger number of minutes, then removed. In some embodiments, removal is by rinsing. In other exemplary embodiments of the invention, removal is by peeling. In some exemplary embodiments of the invention, the composition includes at least 60% magnesium hydroxide. In other exemplary embodiments of the invention, the composition includes various percentages of magnesium hydroxide as set forth above in the description of the composition. Other components of the composition are as set forth above in the description of the composition.

### Exemplary Method of Manufacture

In some exemplary embodiments of the invention there is provided a method of manufacture including heating water and surfactant to form a hot aqueous phase, heating organic components to form a hot organic phase; mixing the hot organic phase into the hot aqueous phase and mixing to homogeneity then cooling slightly and adding at least 45% or at least 50% magnesium hydroxide while mixing. In some exemplary embodiments of the invention, heating of the aqueous and/or organic phases is to 80 C and cooling slightly is to 75 C. In some embodiments, addition of magnesium hydroxide includes adding at least 60% magnesium hydroxide. In some exemplary embodiments of the invention, the surfactant comprises cocamidopropyl betaine. Alternatively or additionally, in some embodiments the organic components include Glyceryl Stearate SE and/or Cetearyl Alcohol and/or Cetearyl Glucoside and/or Ceto-stearyl alcohol and/or Glycerin and/or Jojoba oil. In some exemplary embodiments of the invention, 2 or 3 or more of the organic components are included. Alternatively or additionally, in some embodiments the method includes adding one or more additives such as Phenoxyethanol and/or Ethylhexylglycerin and/or Aloe vera gel. In some exemplary embodiments of the invention, 2 or all 3 of these additives are included.

In some exemplary embodiments of the invention, the resultant facial mask composition is packaged in wide mouth jars.

### Exemplary Dry Mix Composition

In some exemplary embodiments of the invention, a powdered dry mix is provided to be mixed with water by a user prior to use. In some embodiments, a dry mix has a longer shelf life than a “ready to use” formulation including water and/or lipids. In some embodiments, the dry mix is formulated to produce a peel off mask when mixed with water.

In some exemplary embodiments of the invention there is provided a peel off facial mask composition dry mix comprising at least 45% magnesium hydroxide; and other dry ingredients. In some exemplary embodiments of the invention, the dry mix is devoid of lipids. In some exemplary embodiments of the invention, the dry mix includes at least 60% magnesium hydroxide. In other exemplary embodiments of the invention, the percentages of magnesium hydroxide are as set forth under “Exemplary ready to use composition” hereinabove.

In some exemplary embodiments of the invention, the peel off facial mask composition dry mix according includes an alginate (e.g. sodium alginate).

Alternatively or additionally, in some embodiments the peel off facial mask composition dry mix includes calcium sulfate dihydrate and/or Kelzan and/or Disodium EDTA and/or pentasodium triphosphate and/or another chelating agent. In some embodiments, the dry mix includes 2 or all 3 of these ingredients.

Alternatively or additionally, in some embodiments the peel off facial mask composition dry mix includes microcrystalline cellulose (MCC) and/or silica (e.g. MSS 500).

Alternatively or additionally, in some embodiments the peel off facial mask composition dry mix includes magnesium hydroxide which is at least 95% pure. According to various exemplary embodiments of the invention the magnesium hydroxide has a purity value as set forth above in “Exemplary magnesium hydroxide characteristics”.

Alternatively or additionally, in some embodiments the peel off facial mask composition dry mix includes magnesium hydroxide which exhibits a maximum loss on drying (MLOD) of ≤2%. According to various exemplary embodiments of the invention the magnesium hydroxide has a MLOD value as set forth above in “Exemplary magnesium hydroxide characteristics”.

Alternatively or additionally, in some embodiments the peel off facial mask composition dry mix includes magnesium hydroxide which exhibits a loss on ignition (LOI) of 27 to 35%. According to various exemplary embodiments of the invention the magnesium hydroxide has a LOI value as set forth above in “Exemplary magnesium hydroxide characteristics”.

Alternatively or additionally, in some embodiments the peel off facial mask composition dry mix includes magnesium hydroxide which has a bulk density (untapped) of 0.7 g/cc to 1.2 g/cc. According to various exemplary embodiments of the invention the magnesium hydroxide has a bulk density as set forth above in “Exemplary magnesium hydroxide characteristics”.

Alternatively or additionally, in some embodiments the peel off facial mask composition dry mix includes magnesium hydroxide which has a particle size characterized by a residue on 325 mesh (wet sieve) of ≤12%. According to various exemplary embodiments of the invention the magnesium hydroxide has a particle size as set forth above in “Exemplary magnesium hydroxide characteristics”.

In some exemplary embodiments of the invention, peel off facial mask composition dry mix according as described above is provided diluted with water to contain at least 10% magnesium hydroxide. Alternatively or additionally, in some embodiments a peel off facial mask composition dry mix as described above is diluted with water to contain at least 1.7% alginate.

### Additional Exemplary Method of Use

In some exemplary embodiments of the invention there is provided a method comprising mixing a dry mix containing at least 45% magnesium hydroxide with water at a ratio of dry mix:water between 2:3 and 2:7 and applying the resultant mixture to facial skin as a peel off mask. In some exemplary embodiments of the invention, the ratio of dry mix:water is between 2:4.5 and 2:5.5. Alternatively or additionally, in some embodiments the method includes peeling the mask off of said facial skin.

It is expected that during the life of this patent many dermatologically acceptable excipients, diluents and additives will be developed and the scope of the invention is intended to include all such new technologies a priori.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and are not intended to limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of a rinse mask and peel off mask but might also be used as a fabric mask.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

The following materials and methods are used in performance of experiments described in examples hereinbelow:
DEHYTON® K was purchased from BASF.
Cutina GMS was purchased from SE, BASF.
MONTANOV™ 68 MB was purchased from SEPPIC.
PALMEROL®1618 NF was purchased from KLK OLEO.
Jojoba oil was purchased from Jojoba Hazerim (Israel).
CAREMAG® M, ICL was procured from Specialty Minerals
EUXYL® PE 9010 was purchased from Ashland Aloe Vera gel—Tri-K.

Skin sebum content: The sebum measurement was based on the internationally recognized SEBUMETER® method (Sebumeter 815, Courage+Khazaka GmbH). The measurement principle is the photometric method, the grease spot photometer.

According to this method the sebum on skin is collected on a 64 mm2 mat synthetic tape contained in a cassette, then the measuring head of the cassette is inserted into the aperture of the device, where a photocell measures the transparency. The light transmission represents the sebum content on the surface of the measuring area. A microprocessor calculates the result, which is shown on the display in μg sebum/cm² of the skin.

Skin profilometry—pores diameter: Skin surface was quantitatively assessed by Primos 3D (GF Messtechnik GmbH). Primos 3D is a non-contact in vivo skin measurement device based on structured light projection. In conjunction with a comprehensive 3-D measurement and evaluation software, the sensor allows to evaluate skin surface properties (i.e. wrinkle depth, volume, roughness, pores size etc.). In this study pore size is calculated.

### Statistical Analysis:

The instrumental data were analyzed using paired Student t test (intra-group analysis vs TO); while clinical data were analyzed by Wilcoxon signed test (intra-group analysis vs TO).

Differences were considered statistically significant when the p value was <0.05.

The statistical software used for statistical analysis is: NCSS 10—PROFESSIONAL, vers. 10.0.7 released on Jul. 22, 2015 running on Windows Server 2008 R2 Standard.

### Interpretation of Results:

The study was designed to demonstrate the test product claim(s) in the current framework proposed by Commission Regulation (EU) No 655/2013. Endpoints are measured using techniques currently accepted in the cosmetic field while biases are minimized by procedure(s) standardization according to ISO 9001.

For purposes of Quality Management data were analyzed and interpreted by skilled technician according to both descriptive and inferential statistical analysis procedures. Due to the lack of reference values in the cosmetic field, statistical significance (for instrumental analysis) and percentage of subjects showing an effect (for clinical/sensorial endpoints) are the primary criterion to evaluate the correspondence between the proposed claim(s) and the study output(s). In particular Intragroup (vs. TO) or intergroup (e.g. active vs. placebo, treated vs non treated) statistical analysis criterion to reject the null hypothesis (no product effect) is set at p<0.05. For clinical evaluations, the positive effect of the product on the measured parameter is confirmed if more than 50% of the subjects register an improvement.

Finally, for the self-assessment questionnaires, the performance and the pleasantness of the product must be perceived by at least 60% of the subjects. Whenever reference values or threshold values exist, those values are used to validate product claim(s).

### Example 1

### Detailed Exemplary Composition

### Rinse Mask Formulation

In order to provide a magnesium hydroxide-based formulation for testing in human subjects, an existing mask formulation based on 40% Kaolin was used as a control. Direct substitution of 40% Mg(OH)₂ for Kaolin produced a mask which collapsed after a very short period of storage. The percentage of Mg(OH)₂ was increased to 60% (with a compensatory decrease in the amount of water) and the formulation showed no significant loss of volume during storage.

The three formulations are presented in table 1. The 60% Mg(OH)₂ formulation was used as a test formulation in trials with human volunteers as described hereinbelow. Various parameters of facial skin response to the test formulation were evaluated. Some parameters were evaluated by objective measurement and other were evaluated subjectively.

The 20 subjects participating in the study were selected under the supervision of a board-certified dermatologist from a panel of healthy subjects. All 20 subjects were healthy Caucasian females over the age of 18 (mean age 42; range 20-55) with oily skin, dilated pores and slight redness of the facial skin. Subjects that had recently participated in similar studies, or were using products with similar characteristics as the tested product, were excluded.

An initial assessment of each subject was conducted at time zero (TO) prior to administration of the tested product.

For short term tests, clinical evaluations of normalizing effect and skin smoothness performed by the dermatologist 30 minutes (T30 min) after mask removal (first product application).

For long term tests, the test product was administered twice per week for 28 days. At T28 days an instrumental check of the parameters under study (skin sebum content, pores diameter) and clinical evaluation of skin redness, normalizing effect, astringent effect on pores, skin smoothness and distribution of Protoporfinrin IX was conducted.

TABLE 1
initial formulations for rinse mask

| Composition | | Kaolin | Magnesium | Magnesium |
|---|---|---|---|---|
| | Commercial | control | Hydroxide | Hydroxide |
| INCI name | name | Wt % | | |
| Water | Water | 44.1 | 44.1 | 24.1 |
| Cocamidopropyl | Dehyton ® K | 1.5 | 1.5 | 1.5 |
| Betaine (35%) | | | | |
| Glyceryl Stearate | Cutina GMS-SE | 1.0 | 1.0 | 1.0 |
| SE | | | | |
| Cetearyl Alcohol | MONTANOV ™ | 2.0 | 2.0 | 2.0 |
| (and) Cetearyl | 68 MB | | | |
| Glucoside | | | | |
| Ceto-stearyl | Palmerol ® | 1.0 | 1.0 | 1.0 |
| alcohol | 1618 NF | | | |
| Glycerin | Glycerin | 9.0 | 9.0 | 9.0 |
| Jojoba oil | Jojoba oil | 0.3 | 0.3 | 0.3 |
| Kaolin | | 40 | — | — |
| Magnesium | CareMag ® M | — | 40 | 60 |
| Hydroxide | | | | |
| Phenoxyethanol & | Euxyl ® PE 9010 | 1 | 1 | 1 |
| Ethylhexylglycerin | | | | |
| Aloe Vera gel | Aloe Vera gel | 0.1 | 0.1 | 0.1 |

In addition, after first product use (T30 min) and at the end of the study (T28 days), volunteers completed a subjective self-assessment questionnaire in which they ranked various parameters on a numerical scale.

### Example 2

### Effect of 60% Mg(OH)₂ Rinse Mask on Skin Sebum Content

In order to evaluate the effect of the 60% Mg(OH)₂ rinse mask on healthy human subjects, skin sebum content was measured at TO and at T28 days using the method described hereinabove in materials and methods.

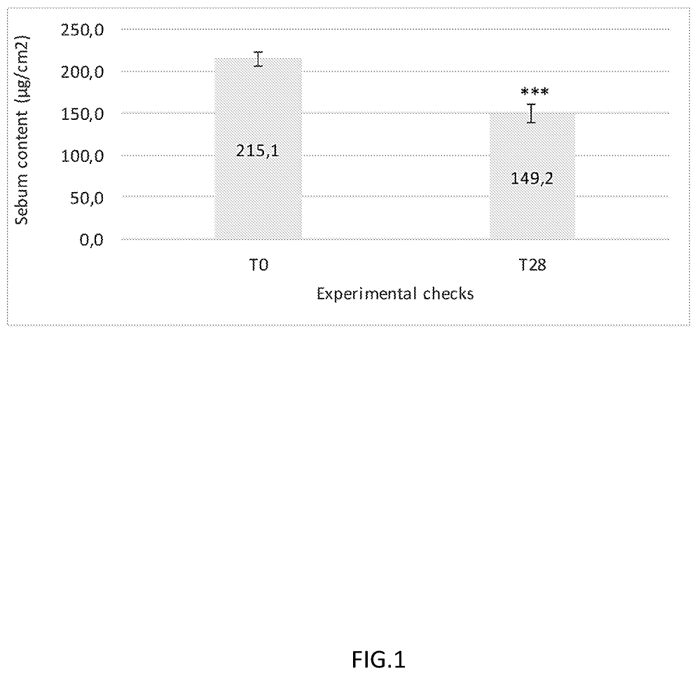

Results are summarized graphically in FIG. 1. The tested facial mask formulation reduced skin sebum content from 215 μg/cm² to 149 μg/cm² and the results were statistically significant.

This example illustrates that the tested facial mask formulation had a positive effect on an important objective parameter of skin quality.

### Example 3

### Effect of 60% Mg(OH)₂ Rinse Mask on Pore Diameter

In order to evaluate the effect of the 60% Mg(OH)₂ rinse mask on healthy human subjects, pore diameter was measured at TO and at T28 days using the method described hereinabove in materials and methods.

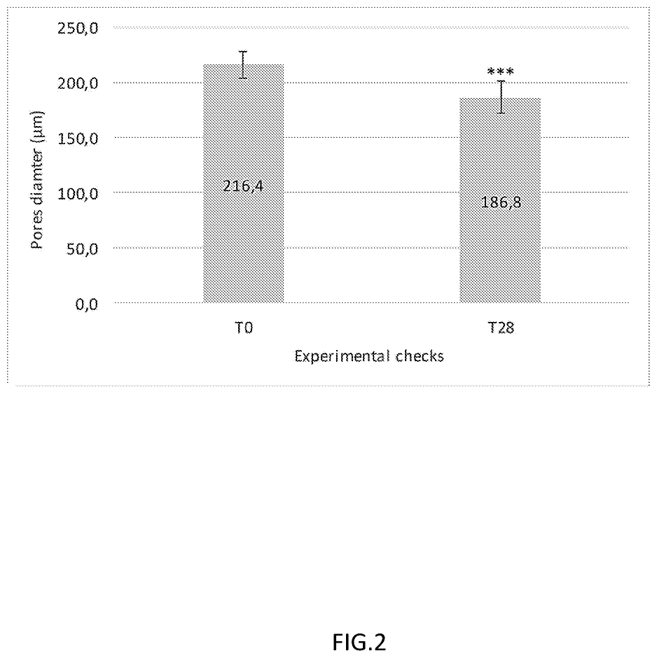

Results are summarized graphically in FIG. 2. The tested facial mask formulation reduced pore diameter from 216 μm to 187 μm and the results were statistically significant.

This example illustrates that the tested facial mask formulation had a positive effect on another important objective parameter of skin quality.

### Example 4

### Effect of 60% Mg(OH)₂ Rinse Mask on Skin Redness

In order to evaluate the effect of the 60% Mg(OH)₂ rinse mask on healthy human subjects, skin redness was subjectively evaluated at T0 and at T28 days using a clinical evaluation questionnaire as described hereinabove in materials and methods.

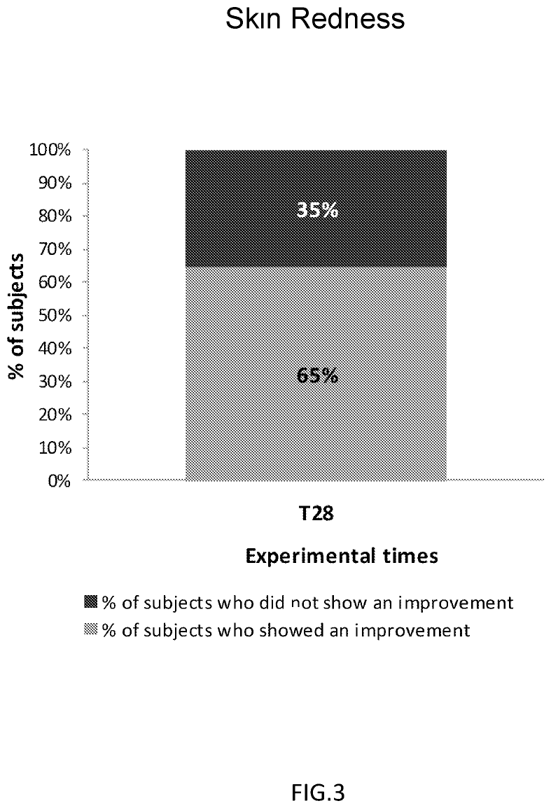

Results are summarized graphically in FIG. 3. The tested facial mask formulation reduced redness in 65% of subjects.

This example illustrates that the tested facial mask formulation had a positive effect on an important subjective parameter of skin quality.

### Example 5

### Normalizing Effect of 60% Mg(OH)₂ Rinse Mask on Facial Skin

In order to evaluate the effect of the 60% Mg(OH)₂ Rinse mask on healthy human subjects, the normalizing effect was subjectively evaluated at T30 min and at T28 days using a clinical evaluation questionnaire as described hereinabove in materials and methods.

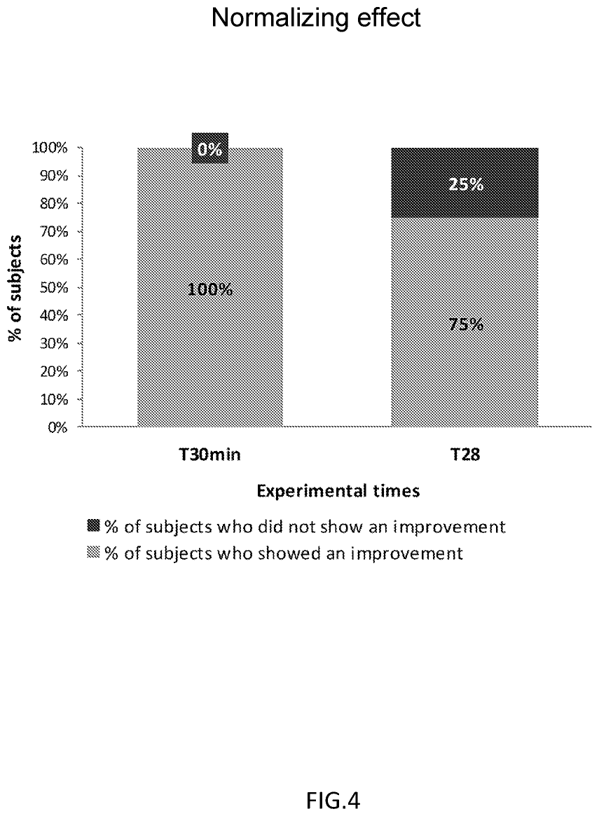

Results are summarized graphically in FIG. 4. The tested facial mask formulation had a normalizing effect in 100% of subjects at T30 min and in 75% of subjects at T28 days.

This example illustrates that the tested facial mask formulation had a positive effect on another important subjective parameter of skin quality and that this effect persisted in 75% of subjects.

### Example 6

### Astringent Effect on Pores of 60% Mg(OH)₂ Rinse Mask

In order to evaluate the effect of the 60% Mg(OH)₂ rinse mask on healthy human subjects, the astringent effect on pores was subjectively evaluated at T28 days using a clinical evaluation questionnaire as described hereinabove in materials and methods.

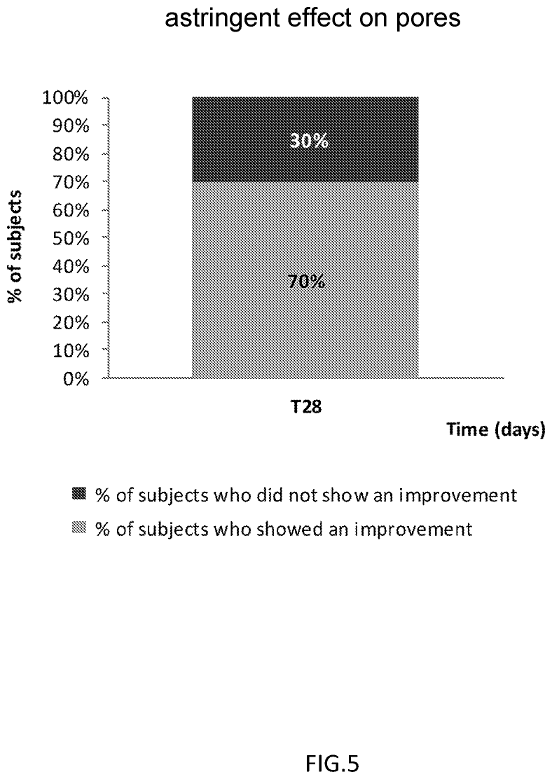

Results are summarized graphically in FIG. 5. The tested facial mask formulation had an astringent effect on pores in 70% of subjects at T28 days.

This example illustrates that the tested facial mask formulation had a positive effect on another important subjective parameter of skin quality.

### Example 7

### Effect of 60% Mg(OH)₂ Rinse Mask on Skin Smoothness

In order to evaluate the effect of the 60% Mg(OH)₂ rinse mask on healthy human subjects, the skin smoothness was subjectively evaluated at T30 min and T28 days using a clinical evaluation questionnaire as described hereinabove in materials and methods.

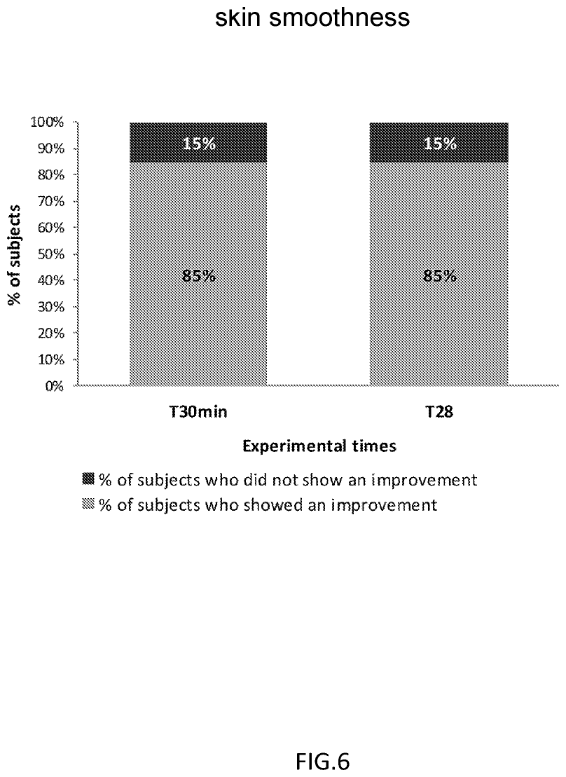

Results are summarized graphically in FIG. 6. The tested facial mask formulation improved skin smoothness in 85% of subjects at T30 Min and in 85% of subjects at T28 days.

This example illustrates that the tested facial mask formulation had a persistent positive effect on skin smoothness which is perhaps the most important parameter in the mind of a typical facial mask user.

### Example 8

### Effect of 60% Mg(OH)₂ Rinse Mask on Protoporfinrin IX Distribution

In order to evaluate the effect of the 60% Mg(OH)₂ rinse mask on healthy human subjects, the Protoporfinrin IX Distribution was subjectively evaluated at T30 min and T28 days using a clinical evaluation questionnaire as described hereinabove in materials and methods.

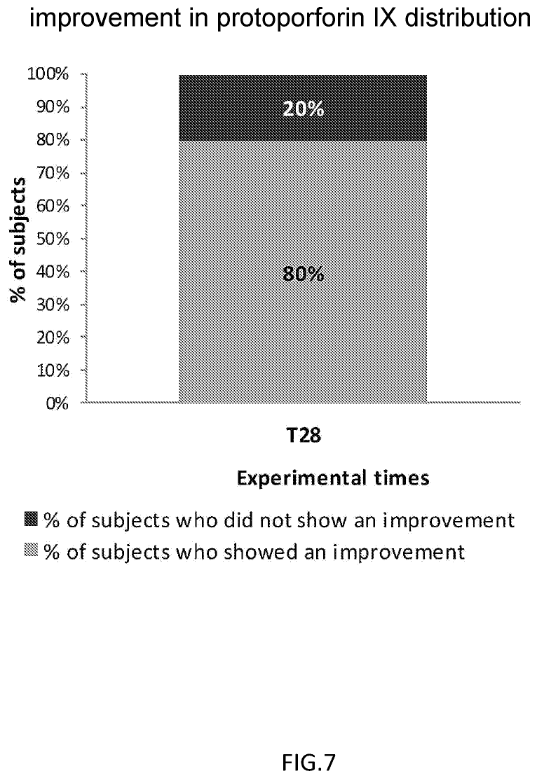

Results are summarized graphically in FIG. 7. The tested facial mask formulation improved Protoporfinrin IX Distribution in 80% of subjects at T28 days.

This example illustrates that the tested facial mask formulation had a persistent positive effect on another important objective parameter.

### Example 9

### Short Term Overall Subject Impression of 60% Mg(OH)₂ Rinse Mask

In order to evaluate the short-term effect of the 60% Mg(OH)₂ rinse mask on healthy human subjects, subjects filled in a self-assessment questionnaire as described hereinabove in materials and methods at T30 min.

Results are summarized in Table 2. The results are expressed as percentage (%) of subjects who expressed the same opinion among those proposed. Positive answers were “agree” and “completely agree”.

The percentages of positive answers establish that the overall impression of the test subjects at T30 Min was favorable.

TABLE 2
Summary of self-assessment questionnaire results at T30 min.

| | | Completely | | | Completely | Positive |
|---|---|---|---|---|---|---|
| No | Question | agree | Agree | Disagree | disagree | answers |
| 1 | The product has a sebum-normalizing effect | 15.0% | 80.0% | 5.0% | 0.0% | 95.0% |
| 2 | My skin is well moisturized | 10.0% | 70.0% | 20.0% | 0.0% | 80.0% |
| 3 | The product helps to reduce the sebum content | 20.0% | 70.0% | 20.0% | 0.0% | 90.0% |
| | on the skin | | | | | |
| 4 | The product helps to reduce pore visibility | 10.0% | 75.0% | 15.0% | 0.0% | 85.0% |
| 5 | My skin is smoother and softer | 20.0% | 80.0% | 0.0% | 0.0% | 100.0% |
| 6 | After mask removal I perceive my skin deeply | 20.0% | 75.0% | 5.0% | 0.0% | 95.0% |
| | purified | | | | | |
| 7 | The mask is gentle on the skin | 20.0% | 65.0% | 15.0% | 0.0% | 85.0% |

### Example 10

### Long Term Overall Subject Impression of 60% Mg(OH)₂ Rinse Mask

In order to evaluate the long-term effect of the 60% Mg(OH)₂ rinse mask on healthy human subjects, subjects filled in a self-assessment questionnaire as described hereinabove in materials and methods at T28 days.

Results are summarized in Table 2. The results are expressed as percentage (%) of subjects who expressed the same opinion among those proposed. Positive answers were “agree” and “completely agree”.

The percentages of positive answers establish that the overall impression of the test subjects at T28 Day was favorable.

The cumulative results of Examples 1 through 10 indicate that the tested 60% Mg(OH)₂ rinse facial mask was well tolerated, effective and made a favorable impression on the test subjects.

TABLE 3
Summary of self-assessment questionnaire results at T28 day.

| | | Completely | | | Completely | Positive |
|---|---|---|---|---|---|---|
| No. | Question | agree | Agree | Disagree | disagree | answers |
| 1 | The product has a sebum-normalizing effect | 20.0% | 75.0% | 5.0% | 0.0% | 95.0% |
| 2 | My skin is well moisturized | 10.0% | 65.0% | 20.0% | 5.0% | 75.0% |
| 3 | After mask removal I perceive my skin deeply | 40.0% | 45.0% | 15.0% | 0.0% | 85.0% |
| | purified | | | | | |
| 4 | The product helps to reduce the sebum content | 35.0% | 55.0% | 5.0% | 5.0% | 90.0% |
| | on the skin | | | | | |
| 5 | My skin is less oily | 30.0% | 60.0% | 10.0% | 0.0% | 90.0% |
| 6 | The skin redness is reduced | 15.0% | 60.0% | 25.0% | 0.0% | 75.0% |
| 7 | Pores appear less visible | 25.0% | 55.0% | 15.0% | 5.0% | 80.0% |
| 8 | The mask is gentle on the skin visible | 30.0% | 45.0% | 20.0% | 5,0% | 75.0% |
| 9 | Is this product better from the regular one that | 20.0% | 55.0% | 25.0% | 0.0% | 75.0% |
| | you are using? | | | | | |
| 10 | My skin is smoother and softer | 30.0% | 50.0% | 20.0% | 0.0% | 80.0% |
| 11 | Do you see a reduction in blemish, blackheads | 20.0% | 60.0% | 20.0% | 0.0% | 80.0% |
| | and other impurities? | | | | | |
| 12 | The product has a bleaching effect (in terms of | 20.0% | 55.0% | 25.0% | 0.0% | 75.0% |
| | redness reduction and more uniform complexion) | | | | | |
| No. Question | | Yes | No | — | — | Positive |
| | | | | | | answers |
| 13 | Is the product well tolerated? | 100.0% | 0.0% | — | — | 100.0% |

### Example 11

### Detailed Exemplary Composition

### Peel Off Mask Formulation

In order to evaluate peel off mask formulations, dry powder pre-mixes are being prepared as set forth in table 4.

TABLE 4
Dry powder pre mixes for peel of masks.

| Ingredient | Wt % | % range |
|---|---|---|
| Magnesium hydroxide (CareMag M) | 64.0% | 45-70 |
| Calcium sulfate dihydrate | 12.5% | 10-14 |
| Sodium Alginate | 12.1% | 9-13 |
| Kelzan | 3.1% | 1-5 |
| Disodium ETDA | 1.8% | 0.5-2.5 |
| MSS 500 (silica) | 6.2% | 4-8 |
| MCC (microcrystalline cellulose) | 0% | 0-4 |

The powdered components listed in the table are mixed together using standard laboratory or industrial equipment to produce a pre-mix. The pre-mix is blended with water in 1:3 (weight ratio powder:water) just before application onto the skin. According to various exemplary embodiments of the invention the ratio varies from 1:2 to 1:4. The peel off mask will be evaluated experimentally as described for the rinse mask in examples 2 through 10 hereinabove. Since the active ingredient is the same, and its amount is similar, as in the rinse mask formulation, similar results are expected.

1. A facial mask composition comprising:
(a) at least 45% magnesium hydroxide;
(b) not more than 30% water; and
(c) not more than 12% lipids.

2. The facial mask composition according to claim 1, comprising at least 60% magnesium hydroxide.

3. The facial mask composition according to claim 1, comprising a surfactant.

4. The facial mask composition according to claim 1, comprising at least one member of the group consisting of Glyceryl Stearate SE, Cetearyl Alcohol, Cetearyl Glucoside, Ceto-stearyl alcohol, Glycerin, and Jojoba oil.

5. The facial mask composition according to claim 1, comprising at least one member of the group consisting of Phenoxyethanol, Ethylhexylglycerin, and Aloe vera gel.

6-11. (canceled)

12. The method comprising:
applying a composition according to claim 1 to facial skin of a subject.

13. The method according to claim 12, wherein said composition comprises at least 60% magnesium hydroxide.

14. A method comprising:
(a) heating water and surfactant to form a hot aqueous phase;
(b) heating organic components to form a hot organic phase; and
(c) mixing the hot organic phase with the hot aqueous phase and mixing to homogeneity then cooling slightly and adding at least 50% magnesium hydroxide while mixing.

15. The method according to claim 14, wherein said adding at least 50% magnesium hydroxide comprises adding at least 60% magnesium hydroxide.

16. The method according to claim 14, wherein said surfactant comprises Cocamidopropyl Betaine.

17. The method according to claim 14, wherein said organic components comprise at least one member of the group consisting of Glyceryl Stearate SE, Cetearyl Alcohol, Cetearyl Glucoside, Ceto-stearyl alcohol, Glycerin, Jojoba oil.

18. The method according to claim 14, comprising adding one or more additives selected from the group consisting of Phenoxyethanol, Ethylhexylglycerin, and Aloe vera gel.

19. The method according to claim 14, comprising packaging the resultant facial mask composition in wide mouth jars.

20. A peel off facial mask composition dry mix comprising:
(a) at least 45% magnesium hydroxide; and
(b) other dry ingredients.

21. The peel off facial mask composition dry mix according to claim 20, comprising at least 60% magnesium hydroxide.

22. The peel off facial mask composition dry mix according to claim 20, comprising an alginate.

23. The peel off facial mask composition dry mix according to claim 20, comprising at least one member of the group consisting of calcium sulfate dihydrate, Kelzan, Disodium EDTA and pentasodium triphosphate.

24. The peel off facial mask composition dry mix according to claim 20, comprising microcrystalline cellulose (MCC) and/or silica.

25. The peel off facial mask composition dry mix according to claim 20, wherein said magnesium hydroxide is at least 95% pure.

26. The peel off facial mask composition dry mix according to claim 20, wherein said magnesium hydroxide exhibits a maximum loss on drying (MLOD) of ≤2%.

27-34. (canceled)
